(19) United States
(12) Patent Application Publication
(10) Pub. No.: US 2012/0287524 A1
(43) Pub. Date: Nov. 15, 2012

(54) Novel Compound, Pigment Dispersion Composition and Photosensitive Resin Composition Including the Same, and Color Filter Using the Same

(75) Inventors: Seong-Yong Uhm, Uiwang-si (KR); Seong-Ryong Nam, Uiwang-si (KR); Taek-Jin Baek, Uiwang-si (KR); Tae-Gyu Chun, Uiwang-si (KR); Kyung-Hee Hyung, Uiwang-si (KR)

(73) Assignee: CHEIL INDUSTRIES INC., Gumi-si (KR)

(21) Appl. No.: 13/503,918

(22) PCT Filed: Jun. 22, 2010
(86) PCT No.: PCT/KR10/04046

§ 371 Date: Apr. 25, 2012

(30) Foreign Application Priority Data
Dec. 18, 2009 (KR) 10-2009-0127309

(51) Int. Cl. C07D 403/14; C07F 1/08; G02B 5/22; C07D 209/64; G03F 7/032; C07D 251/54; C07D 401/10

(52) U.S. Cl. 359/891; 544/198; 544/197; 540/134; 546/167; 548/450; 430/281.1; 430/283.1

(57) ABSTRACT

Disclosed are a novel compound, a pigment dispersion composition including the same, a photosensitive resin composition including the same, and a color filter using the same.

### TECHNICAL FIELD

This disclosure relates to a novel compound, a pigment dispersion composition, and a photosensitive resin composition including the same, and a color filter using the same.

### BACKGROUND ART

Recently, as large-screen liquid crystal displays (LCD) have spread, improved performance of an LCD has increasingly been required. Since a color filter as a part of the LCD is the most important part for displaying a color, researchers have continuously undertaken studies on improving a process margin for productivity. In addition, in order to increase color purity in the case of a large-screen LCD, a colorant concentration for a photosensitive resin composition for preparing a color filter has been increased, and in order to increase productivity and yield during a manufacturing process, a photosensitive resin composition having a low developing rate and excellent sensitivity even in a small exposure amount has been required.

A color filter is fabricated using a pigment dispersion method that forms a pattern using a photosensitive resin composition with dispersed pigment particulates as a colorant. During the manufacturing process for the color filter using the pigment dispersion method, in order to maintain a pattern formed through several chemical treatments, a photosensitive resin composition having increased development margins and being capable of improving yields for a color filter due to chemical resistance is required. For this purpose, a pigment dispersant using for pigment dispersion and a binder resin should be controlled.

Generally, a pigment dispersant is used for maintaining a good dispersion phase for a pigment. The pigment dispersant includes an adsorption moiety with a pigment and an affinity moiety with a solvent as a dispersion medium, and has a role of a dispersant through the balance of the two moieties. Herein, when a pigment has an acidic surface, the pigment dispersant has a base pigment adsorption moiety, while when a pigment has a base surface, the pigment dispersant has an acidic pigment adsorption moiety. As for fabricating a color filter, a problem of decreasing developing margins may be caused in the former case, and pattern characteristics of color pixels may be decreased in the latter case.

### DISCLOSURE OF INVENTION

### Technical Problem

One embodiment provides a novel compound that can provide a pigment dispersion composition having high dispersibility and storage stability in a small amount.

Another embodiment provides a pigment dispersion composition including the novel compound as a pigment dispersant.

Yet another embodiment provides a photosensitive resin composition including the pigment dispersion composition.

Still another embodiment provides a color filter using the photosensitive resin composition having excellent color characteristics such as luminance, contrast ratio, and the like, and excellent pattern characteristics.

### Solution to Problem

According to an embodiment, a compound represented by the following Chemical Formula 1 is provided.

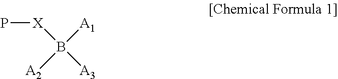

In Chemical Formula 1,

B is a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, or a substituted or unsubstituted C6 to C30 arylene group,

A₁ to A₃ are independently a substituent represented by the following Chemical Formula 3, CH₂ COOH, COOH, or SO₃H,

X is CONH, and

P is one of substituents represented by the following Chemical Formulae 4-1 to 4-4.

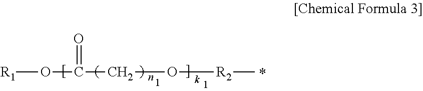

In Chemical Formula 3,

R₁ is a substituent derived from a compound including a hydroxy group,

R₂ is CO or SO₂,

n₁ is an integer ranging from 1 to 6, and

k₁ is an integer ranging from 1 to 20.

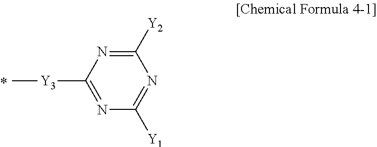

In Chemical Formula 4-1,

Y₁ and Y₂ are independently hydrogen, a halogen, a hydroxy group, a substituted or unsubstituted C1 to C20 alkoxy group, a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C3 to C20 cycloalkynyl group, a substituted or unsubstituted C2 to C20 heterocycloalkyl group, a substituted or unsubstituted C2 to C20 heterocycloalkenyl group, a substituted or unsubstituted C2 to C20 heterocycloalkynyl group, a substituted or unsubstituted C6 to C30 aryl group, a substituted or unsubstituted C6 to C30 aryloxy group, an amine group including a substituted or unsubstituted C1 to C20 alkyl group or a C6 to C30 aryl group, or an amine group including a substituted or unsubstituted C1 to C20 alkyl group or a C6 to C30 aryl group, and

Y₃ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group.

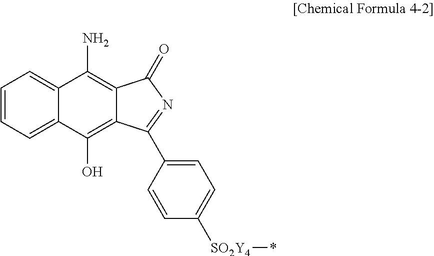

In Chemical Formula 4-2,

Y₄ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group.

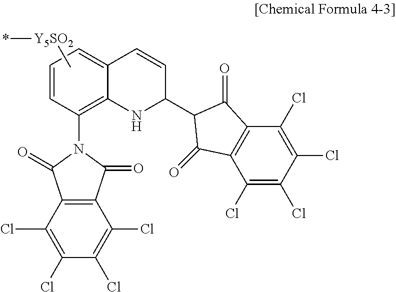

In Chemical Formula 4-3,

Y₅ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group.

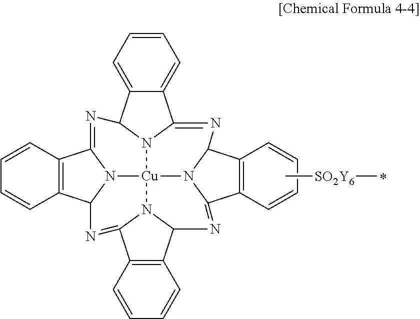

In Chemical Formula 4-4,

Y₆ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group.

The compound of Chemical Formula 1 may have a weight average molecular weight ranging from about 3,000 to about 15,000.

According to another embodiment, a pigment dispersant composition is provided that includes (A) a pigment, (B) a pigment dispersant including a compound represented by Chemical Formula 1, (C) a binder resin, and (D) a solvent.

The pigment dispersion composition may include about 5 to about 40 wt % of the pigment (A), about 1 to about 20 wt % of the pigment dispersant (B), about 1 to about wt % of the binder resin (C), and a balance amount of the solvent (D).

The pigment dispersion composition may have a viscosity ranging from about 1 to about 50 cps.

According to yet another embodiment, a photosensitive resin composition including the pigment dispersion composition is provided.

According to still another embodiment, a color filter fabricated by using the photosensitive resin composition is provided.

Further embodiments will be described in detail.

### Advantageous Effects of Invention

The novel compound can be used in a pigment dispersion composition having high dispersibility and storage stability in a small amount.

### Best Mode for Carrying Out the Invention

Exemplary embodiments will hereinafter be described in detail. However, these embodiments are only exemplary, and the present invention is not limited thereto.

As used herein, when a specific definition is not otherwise provided, the term “substituted” refers to one substituted with a halogen (F, Cl, Br, or I), a hydroxy group, a C1 to C20 alkoxy group, a C1 to C20 oxyalkylene group, a nitro group, a cyano group, an amino group, an imino group, an azido group, an amidino group, a hydrazine group, a hydrazono group, a carbonyl group, a carbamyl group, a thiol group, an ester group, a carboxyl group or a salt thereof, a sulfonic acid group or a salt thereof, phosphoric acid or a salt thereof, a C1 to C20 alkyl group, a C1 to C20 alkylene group, a C2 to C20 alkenyl group, a C2 to C20 alkenylene group, a C2 to C20 alkynyl group, a C2 to C20 alkynylene group, a C6 to C30 aryl group, a C6 to C30 arylene group, a C3 to C20 cycloalkyl group, a C3 to C20 cycloalkylene group, a C3 to C20 cycloalkenyl group, a C3 to C20 cycloalkenylene group, a C3 to C20 cycloalkynyl group, a C3 to C20 cycloalkynylene group, a C2 to C20 heterocycloalkyl group, a C2 to C20 heterocycloalkylene group, a C2 to C20 heterocycloalkenyl group, a C2 to C20 heterocycloalkenylene group, a C2 to C20 heterocycloalkynyl group, a C2 to C20 heterocycloalkynylene group, or a combination thereof instead of hydrogen. The substituents may be substituted with at least one of the aforementioned substituents instead of hydrogen.

As used herein, when specific definition is not otherwise provided, the terms “heterocycloalkyl group”, “heterocycloalkenyl group”, “heterocycloalkynyl group”, “heterocycloalkylene group”, “heterocycloalkenylene group”, and “heterocycloalkynylene group” refer to a heterocycloalkyl group, a heterocycloalkenyl group, a heterocycloalkynyl group, a heterocycloalkylene group, a heterocycloalkenylene group, and a heterocycloalkynylene group including at least one heteroatom of N, O, S, or P in each of a cyclic compound.

As used herein, when specific definition is not otherwise provided, the term “(meth)acrylate” refers to both “acrylate” and “methacrylate”.

In one embodiment, the compound represented by the following Chemical Formula 1 is provided.

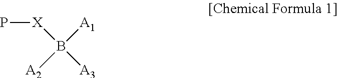

In Chemical Formula 1, B is a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, or a substituted or unsubstituted C6 to C30 arylene group.

Examples of B include linkers represented by the following Chemical Formulae 2-1 to 2-3.

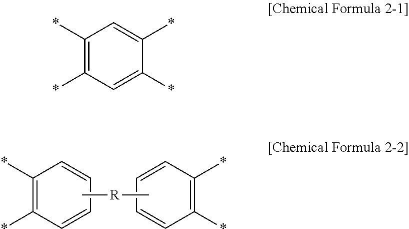

In Chemical Formula 2-2, R may be a C1 to C20 alkylene group, O, CO, or SO₂.

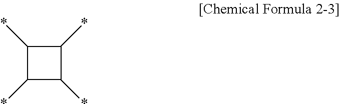

In Chemical Formula 1, A₁ to A₃ may independently be a substituent represented by the following Chemical Formula 3, CH₂ COOH, COOH, or SO₃H. Herein, the substituent represented by the following Chemical Formula 3 is an affinity moiety for a solvent for compatibility with a solvent, and CH₂ COOH, COOH, or SO₃H is a soluble moiety through an alkaline developing process. At least two of A₁ to A₃ may be the same substituents. The substituent represented by the following Chemical Formula 3 may have a weight average molecular weight ranging from about 100 to about 10,000 g/mol.

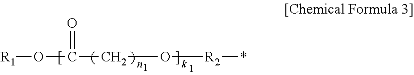

In Chemical Formula 3,

R₁ is a substituent derived from a compound including a hydroxy group,

R₂ is CO or SO₂,

n₁ is an integer ranging from 1 to 6, and

k₁ is an integer ranging from 1 to 20.

As for the compound corresponding to R₁ of Chemical Formula 3, anything that includes a hydroxy group at its terminal end may be used. Examples of the compound include: an aliphatic alcohol such as methanol, ethanol, propanol, heptanol, octanol, isooctanol, decanol, or dodecanol; an aromatic alcohol such as benzyl alcohol, or phenoxy ethanol; an alkylene glycol ether such as ethylene glycol methylether, ethylene glycol ethylether, ethylene glycol butylether, propylene glycol methylether, diethylene glycol ethylether, or diethylene glycolbutyl ether; and hydroxyethyl(meth)acrylate, hydroxypropyl(meth)acrylate, hydroxyphenoxypropyl(meth)acrylate, hydroxy(meth)acryloyloxypropyl(meth)acrylate, or glycerine di(meth)acrylate, including an unsaturated double bond.

In Chemical Formula 1, X may be CONH.

P of Chemical Formula 1 is an affinity moiety for a pigment that can be adsorbed on the surface of a pigment particle, it has the same or a partially similar structure with a pigment compound, and it may be adsorbed through a π-π molecular interaction. P may be one of substituents represented by the following Chemical Formulae 4-1 to 4-4.

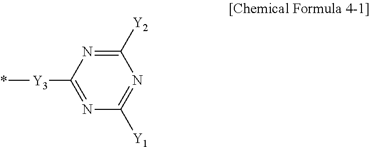

In Chemical Formula 4-1, Y₁ and Y₂ are independently hydrogen, a halogen, a hydroxy group, a substituted or unsubstituted C1 to C20 alkoxy group, a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C3 to C20 cycloalkynyl group, a substituted or unsubstituted C2 to C20 heterocycloalkyl group, a substituted or unsubstituted C2 to C20 heterocycloalkenyl group, a substituted or unsubstituted C2 to C20 heterocycloalkynyl group, a substituted or unsubstituted C6 to C30 aryl group, a substituted or unsubstituted C6 to C30 aryloxy group, an amine group including a substituted or unsubstituted C1 to C20 alkyl group or a C6 to C30 aryl group, or an amine group including a substituted or unsubstituted C1 to C20 alkyl group or a C6 to C30 aryl group.

In one embodiment, Y₁ and Y₂ are independently hydrogen, a hydroxy group, a methyl group, an ethyl group, a propyl group, a butyl group, an isobutyl group, an amyl group, a hexyl group, an ethylhexyl group, a heptyl group, an octyl group, an isooctyl group, a nonyl group, a decyl group, a dodecyl group, a hexadecyl group, a octadecyl group, a cyclopropyl group, a cyclopentyl group, a cyclohexyl group, an allyl group, a methoxy group, a hydroxyethyl group, a methoxyethyl group, a 2-hydroxypropyl group, a methoxypropyl group, a cyanoethyl group, an ethoxy group, a butoxy group, a hexyloxy group, a methoxyethoxyethyl group, a methoxyethoxyethoxyethyl group, a hexamethyleneimine group, a morpholino group, a piperidine group, a piperazine group, an ethylenediamine group, a propylenediamine group, a hexamethylenediamine group, a triethylenediamine group, a pyrrole group, a imidazole group, a pyridine group, a carboxylmethyl group, a trimethoxysilylpropyl group, a triethoxysilylpropyl group, a phenyl group, a methoxyphenyl group, a cyanophenyl group, a phenoxy group, a tolyl group, a benzyl group, an aminobenzoimidazolidone group and derivatives thereof, a polyallylamine group and derivatives thereof, a polyethyleneimine group, derivatives thereof, and the like.

In Chemical Formula 4-1, Y₃ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group. Y₃ is linked to X of Chemical Formula 1. In one embodiment, Y₃ is a single bond, a methylene group, an ethylene group, a propylene group, a butylene group, an isobutylene group, an amylene group, a hexylene group, an ethylhexylene group, a heptylene group, an octylene group, an isooctylene group, a nonylene group, a decylene group, a dodecylene group, a hexadecylene group, a octadecylene group, a cyclopropylene group, a cyclopentylene group, a cyclohexylene group, an allylene group, a oxymethylene group, a hydroxyethylene group, a methoxyethylene group, a 2-hydroxypropylene group, a methoxypropylene group, a cyanoethylene group, an oxyethylene group, a oxybutylene group, a oxyhexylene group, a methoxyethoxyethylene group, a methoxyethoxyethoxyethylene group, a hexamethyleneimine group, a morpholino group, a piperidine group, a piperazine group, an ethylenediamine group, a propylenediamine group, a hexamethylenediamine group, a triethylenediamine group, a pyrrole group, a imidazole group, a pyridine group, a carboxylmethylene group, a trimethoxysilylpropylene group, a triethoxysilylpropylene group, a phenylene group, a methoxyphenylene group, a cyanophenylene group, a oxyphenylene group, a tolylene group, a benzylene group, an aminobenzoimidazolidone group and derivatives thereof, a polyallylamine group and derivatives thereof, a polyethyleneimine group, derivatives thereof, and the like.

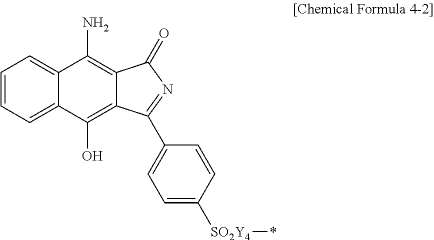

In Chemical Formula 4-2, Y₄ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group. In one embodiment, Y₄ is a single bond, a methylene group, an ethylene group, a propylene group, a butylene group, an isobutylene group, an amylene group, a hexylene group, an ethylhexylene group, a heptylene group, an octylene group, an isooctylene group, a nonylene group, a decylene group, a dodecylene group, a hexadecylene group, a octadecylene group, a cyclopropylene group, a cyclopentylene group, a cyclohexylene group, an allylene group, a oxymethylene group, a hydroxyethylene group, a methoxyethylene group, a 2-hydroxypropylene group, a methoxypropylene group, a cyanoethylene group, an oxyethylene group, a oxybutylene group, a oxyhexylene group, a methoxyethoxyethylene group, a methoxyethoxyethoxyethylene group, a hexamethyleneimine group, a morpholino group, a piperidine group, a piperazine group, an ethylenediamine group, a propylenediamine group, a hexamethylenediamine group, a triethylenediamine group, a pyrrole group, a imidazole group, a pyridine group, a carboxylmethylene group, a trimethoxysilylpropylene group, a triethoxysilylpropylene group, a phenylene group, a methoxyphenylene group, a cyanophenylene group, a oxyphenylene group, a tolylene group, a benzylene group, an aminobenzoimidazolidone group and derivatives thereof, a polyallylamine group and derivatives thereof, a polyethyleneimine group, derivatives thereof, and the like.

The substituent represented by the Chemical Formula 4-2 may be obtained by a pigment red 255 that is a kind of pigment.

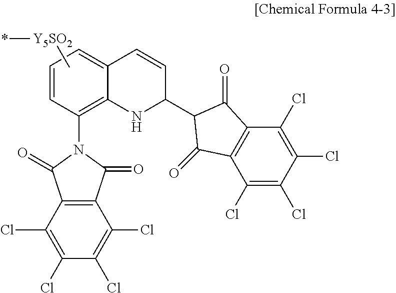

In Chemical Formula 4-3, Y₅ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group. In one embodiment, Y₅ is a single bond, a methylene group, an ethylene group, a propylene group, a butylene group, an isobutylene group, an amylene group, a hexylene group, an ethylhexylene group, a heptylene group, an octylene group, an isooctylene group, a nonylene group, a decylene group, a dodecylene group, a hexadecylene group, a octadecylene group, a cyclopropylene group, a cyclopentylene group, a cyclohexylene group, an allylene group, a oxymethylene group, a hydroxyethylene group, a methoxyethylene group, a 2-hydroxypropylene group, a methoxypropylene group, a cyanoethylene group, an oxyethylene group, a oxybutylene group, a oxyhexylene group, a methoxyethoxyethylene group, a methoxyethoxyethoxyethylene group, a hexamethyleneimine group, a morpholino group, a piperidine group, a piperazine group, an ethylenediamine group, a propylenediamine group, a hexamethylenediamine group, a triethylenediamine group, a pyrrole group, a imidazole group, a pyridine group, a carboxylmethylene group, a trimethoxysilylpropylene group, a triethoxysilylpropylene group, a phenylene group, a methoxyphenylene group, a cyanophenylene group, a oxyphenylene group, a tolylene group, a benzylene group, an aminobenzoimidazolidone group and derivatives thereof, a polyallylamine group and derivatives thereof, a polyethyleneimine group, derivatives thereof, and the like.

The substituent represented by the Chemical Formula 4-3 may be obtained by a pigment yellow 138 that is a kind of pigment.

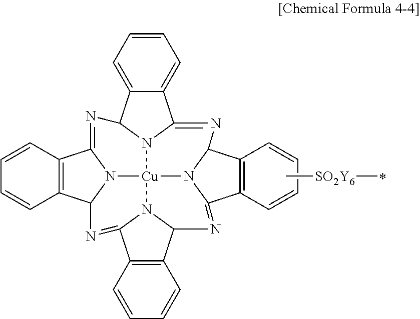

In Chemical Formula 4-4, Y₆ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group. In one embodiment, Y₆ is a single bond, a methylene group, an ethylene group, a propylene group, a butylene group, an isobutylene group, an amylene group, a hexylene group, an ethylhexylene group, a heptylene group, an octylene group, an isooctylene group, a nonylene group, a decylene group, a dodecylene group, a hexadecylene group, a octadecylene group, a cyclopropylene group, a cyclopentylene group, a cyclohexylene group, an allylene group, a oxymethylene group, a hydroxyethylene group, a methoxyethylene group, a 2-hydroxypropylene group, a methoxypropylene group, a cyanoethylene group, an oxyethylene group, a oxybutylene group, a oxyhexylene group, a methoxyethoxyethylene group, a methoxyethoxyethoxyethylene group, a hexamethyleneimine group, a morpholino group, a piperidine group, a piperazine group, an ethylenediamine group, a propylenediamine group, a hexamethylenediamine group, a triethylenediamine group, a pyrrole group, a imidazole group, a pyridine group, a carboxylmethylene group, a trimethoxysilylpropylene group, a triethoxysilylpropylene group, a phenylene group, a methoxyphenylene group, a cyanophenylene group, a oxyphenylene group, a tolylene group, a benzylene group, an aminobenzoimidazolidone group and derivatives thereof, a polyallylamine group and derivatives thereof, a polyethyleneimine group, derivatives thereof, and the like.

The substituent represented by the Chemical Formula 4-4 may be obtained by a pigment blue 15-6 that is a kind of pigment.

The compound of Chemical Formula 1 may be used singularly or in combination of two or more.

The compound of Chemical Formula 1 may have a weight average molecular weight ranging from about 1000 to about 15,000, and in one embodiment, from about 1,000 to about 10,000. When the weight average molecular weight is within the range, dispersion stability is increased due to excellent steric hindrance of a pigment dispersant, and viscosity is decreased by increasing affinity of a solvent.

In another embodiment, a pigment dispersant including a compound represented by Chemical Formula 1 and a pigment dispersion composition including a binder resin and a solvent are provided. Exemplary components included in the pigment dispersion composition will hereinafter be described in detail. However, these embodiments are only exemplary, and the present invention is not limited thereto.

(A) Pigment

As for the pigment, a red pigment, a green pigment, a blue pigment, a yellow pigment, and a violet pigment may be used. Examples of the pigment may include a condensed polycyclic pigment such as an anthraquinone-based pigment and a perylene-based pigment, a phthalocyanine pigment, and an azo-based pigment. These pigments may be used singularly or in combination of two or more.

The pigment may be used by decreasing a primary particle diameter. The methods used for decreasing a primary particle diameter include: decreasing the particle diameter to 70 nm or less by mixing a water-soluble inorganic salt and a wetting agent, and then kneading in a high pressure kneader; an acid pasting method that includes dissolving pigments in acid such as sulfuric acid and precipitating by putting them in a poor solvent to obtain small particles; or dry grinding pigments for a long time using a high speed sand mill, but are not limited thereto. As for the pigment, a commercially available pigment having a decreased primary particle diameter may be used.

The pigment may have a primary particle diameter (D50) ranging from 30 to 70 nm. When the pigment has a primary particle diameter within the range, heat resistance, chemical resistance, durability, and the like are improved after forming a pixel.

The pigment may be used in an amount ranging from about 5 to about 40 wt % based on the total amount of the pigment dispersion composition, and in one embodiment, it may be used in an amount ranging from about 10 to about 30 wt %. When the pigment is used within the range, color reproducibility may be improved due to improving colorfastness, and therefore development properties and stability are increased.

(B) Pigment Dispersant

As for the pigment dispersant, a compound represented by Chemical Formula 1 may be used.

The pigment dispersant may be used in an amount ranging from about 1 to about 20 wt % based on the total amount of the pigment dispersion composition, and in one embodiment, it may be used in an amount ranging from about 3 to about 10 wt % based on the total amount of the pigment dispersion composition. When the pigment dispersant is used within the range, dispersion stability and development properties are increased.

(C) Binder Resin

As for the binder resin, an acryl-based copolymer resin may be used. The acryl-based copolymer may be copolymerized using styrene, N-benzylphthalic imide, (meth)acrylic acid, alkyl(meth)acrylate, aryl(meth)acrylate, alcohol (meth)acrylate, alkylaryl(meth)acrylate, or succinic (meth)acrylate as a monomer, and may be used singularly or in a combination thereof.

In one embodiment, the acryl-based copolymer may be copolymerized using acrylic acid, methacrylic acid, methyl(meth)acrylate, ethyl(meth)acrylate, propyl(meth)acrylate, ethylhexyl(meth)acrylate, phenyl(meth)acrylate, acrylic acid benzyl(meth)acrylate, benzyl(meth)acrylate, tolyl(meth)acrylate, o-silyl (meth)acrylate, glycerol (meth)acrylate, alkylaryl(meth)acrylate, or succinic (meth)acrylate as a monomer, and may be used singularly or in a combination.

Further, the binder resin may include an acryl-based copolymer including at least one or more acidic group. The acidic group may include a carboxyl group, a sulfonic acid group, or a phosphoric acid group.

The binder resin may have an acid value ranges from about 70 to about 150 mgKOH/g. The acid value of a binder resin is determined by an amine value of a dispersing agent, and a dispersing agent generally has an amine value ranging from about 10 to about 200 mgKOH/g. The amine value of the dispersing agent is compensated by the acid value of the binder resin, and therefore stability of a pigment dispersion composition is improved. Further when the pigment dispersion composition is for a color photoresist, developing of unexposed parts may be made easier. In order to improve development properties of unexposed parts, not only the acid value of a binder resin added for preparing a photoresist is important, but also the acid of a binder resin used for preparing a dispersion composition is important. Since the dispersing agent has a fairly high amine value, a binder resin having a higher acid value may be preferable, and in one embodiment, the binder resin may have an acid value ranging from about 80 to about 130 mgKOH/g in order to compensate the amine value.

The binder resin may have a weight average molecular weight ranging from about 3,000 to about 50,000, and in one embodiment, it may have a weight average molecular weight ranging from about 5,000 to about 40,000. When the binder resin has a weight average molecular weight ranging from about 3,000 to about 50,000, an excellent balance of dispersion and viscosity may be obtained.

The binder resin may be used in an amount ranging from about 1 to about 20 wt % based on the total amount of the pigment dispersion composition, and in one embodiment, it may be used in an amount ranging from about 3 to about 10 wt %. When the binder resin is used within the range, development properties are improved and surface flatness and smoothness are obtained due to improved cross-linking.

(D) Solvent

Non-limiting examples of the solvent may include: alcohols such as methanol, ethanol, 1-octanol, 1-nonanol, benzyl alcohol, and the like; ethers such as dichloroethyl ether, n-butyl ether, diisoamyl ether, methylphenyl ether, tetrahydrofuran, benzylethylether, dihexylether, and the like; glycol ethers such as ethylene glycol methylether, ethylene glycol ethylether, propylene glycol methylether, and the like; cellosolve acetates such as methyl cellosolve acetate, ethyl cellosolve acetate, diethyl cellosolve acetate, phenyl cellosolve acetate, and the like; carbitols such as methylethyl carbitol, diethyl carbitol, diethylene glycol monomethylether, diethylene glycol monoethylether, diethylene glycol dimethylether, diethylene glycol methylethylether, diethylene glycol diethylether, and the like; propylene glycol alkylether acetates such as propylene glycol methylether acetate, propylene glycol propylether acetate, and the like; aromatic hydrocarbons such as toluene, xylene, and the like; ketones such as methylethylketone, cyclohexanone, 4-hydroxy-4-methyl-2-pentanone, methyl-n-propylketone, methyl-n-butylketone, methyl-n-amylketone, 2-heptanone, and the like; saturated aliphatic monocarboxylic acid alkyl esters such as ethyl acetate, acetic acid-n-butyl, acetic acid isobutyl, and the like; lactic acid esters such as methyl acetate, ethyl lactate, and the like; oxy acetic acid alkyl esters such as oxy acetic acid methyl, oxy ethyl acetate, butyl oxy acetate, and the like; alkoxy acetic acid alkyl esters such as methyl methoxy acetate, methoxy ethyl acetate, butyl methoxy acetate, methyl ethoxy acetate, ethoxy ethyl acetate, and the like; 3-oxy propionic acid alkylesters such as 3-oxy methyl propionate, 3-oxy ethyl propionate, and the like; 3-alkoxy propionic acid alkyl esters such as 3-methoxy methyl propionate, 3-methoxy ethyl propionate, 3-ethoxy ethyl propionate, 3-ethoxy methyl propionate, and the like; 2-oxy propionic acid alkyl esters such as 2-oxy methyl propionate, 2-oxy ethyl propionate, 2-oxy propyl propionate, and the like; 2-alkoxy propionic acid alkyl esters such as 2-methoxy methyl propionate, 2-methoxy ethyl propionate, 2-ethoxy ethyl propionate, 2-ethoxy methyl propionate, and the like; 2-oxy-2-methyl propionic acid esters such as 2-oxy-2-methyl methyl propionate, 2-oxy-2-methyl ethyl propionate, and the like; monooxy monocarboxylic acid alkyl esters of 2-alkoxy-2-methyl propionic acid alkyls such as 2-methoxy-2-methyl methyl propionate, 2-ethoxy-2-methyl ethyl propionate, and the like; esters such as 2-hydroxy ethyl propionate, 2-hydroxy-2-methyl ethyl propionate, hydroxy ethyl acetate, methyl 2-hydroxy-3-methyl butanoate, and the like; ketone acid ester compounds such as ethyl pyruvate and the like; amides such as N-methylformamide, N,N-dimethyl formamide, N-methylacetamide, N,N-dimethyl acetamide, and the like; N-methylformanilide; N-methylpyrrolidone; dimethylsulfoxide; acetylacetone; isophorone; caproic acid; caprylic acid; benzyl acetate; ethyl benzoate; diethyl oxalate; diethyl malate; γ-butyrolactone; or carbonate alkylenes such as carbonate ethylene; carbonate propylene, and the like. These solvents may be used singularly or in a combination of two or more.

In terms of miscibility and reactivity of the solvent, glycol ethers such as ethylene glycol monoethyl ether, and the like; ethylene glycol alkylether acetates such as ethyl cellosolve acetate, and the like; esters such as 2-hydroxy ethyl propionate, and the like; diethylene glycols such as diethylene glycol monomethyl ether, and the like; propylene glycol alkylether acetates such as propylene glycol methylether acetate, propylene glycol propylether acetate, and the like, may be used.

The solvent may be included in a balance amount based on the total amount of the pigment dispersion composition, and in one embodiment, may be included ranging from about 20 to about 80 wt % based on the total amount of the pigment dispersion composition. When the solvent is included within the range, the viscosity of the pigment dispersion composition may be suitably maintained and therefore physical and optical characteristics of articles may be improved when the pigment dispersion composition is applicable to the articles.

The pigment dispersion composition may have a viscosity ranging from about 1 to about 50 cps, in one embodiment, may have a viscosity ranging from about 3 to about cps. When the viscosity is within the range, it may obtain excellent dispersion stability.

There are no specific manufacturing methods for preparing the pigment dispersion composition, but in one embodiment, pigment minimizing is processed by adding a pigment in a matrix including a pigment dispersant, a binder resin, and a solvent, and dispersing it using a dispersing machine such as a bead mill, a ball mill, or a rod mill to prepare the pigment dispersion composition.

In another embodiment, a photosensitive resin composition including the pigment dispersion composition, an alkaline-soluble resin, a photopolymerization initiator, a photopolymerizable monomer, and a solvent is provided. Hereinafter, the components of the photosensitive resin composition are illustrated in detail.

( [symbol] ) Pigment Dispersion Composition

The pigment dispersion composition is the same as described above.

The pigment dispersion composition may be included in an amount ranging from 0.1 to 40 wt % based on the total amount of the photosensitive resin composition, and in one embodiment, may be included in an amount ranging from 5 to 35 wt % based on the total amount of the photosensitive resin composition. When the pigment dispersion composition is included within the range, it may have excellent pattern characteristics and development properties for preparing a color filter.

( [symbol] ) Alkaline-Soluble Resin

The alkaline-soluble resin is a copolymer of the first ethylenic unsaturated monomer and the second ethylenic unsaturated monomer that is able to be copolymerized therebetween, and includes at least one acryl-based repeating unit.

The first ethylenic unsaturated monomer is an ethylenic unsaturated monomer including at least one carboxyl group, and includes acrylic acid, methacrylic acid, maleic acid, itaconic acid, fumaric acid, or a combination thereof.

The first ethylenic unsaturated monomer may be included in an amount ranging from about 10 to about 40 wt % based on the total amount of the alkaline-soluble resin, and in one embodiment, may be included in an amount ranging from about 20 to about 30 wt % based on the total amount of the alkaline-soluble resin.

The second ethylenic unsaturated monomer may include: an aromatic vinyl compound such as styrene, α-methylstyrene, vinyltoluene, vinylbenzylmethylether, and the like; an unsaturated carboxylic acid ester compound such as methyl(meth)acrylate, ethyl(meth)acrylate, butyl(meth)acrylate, 2-hydroxyethyl(meth)acrylate, 2-hydroxy butyl(meth)acrylate, benzyl(meth)acrylate, cyclohexyl(meth)acrylate, phenyl(meth)acrylate, and the like; an unsaturated carboxylic acid amino alkyl ester compound such as 2-aminoethyl(meth)acrylate, 2-dimethylaminoethyl(meth)acrylate, and the like; a carboxylic acid vinyl ester compound such as vinyl acetate, vinyl benzoate, and the like; an unsaturated carboxylic acid glycidyl ester compound such as glycidyl(meth)acrylate, and the like; a vinyl cyanide compound such as (meth)acrylonitrile, and the like; and an unsaturated amide compound such as (meth)acrylamide, and the like, and these may be used singularly or in combination of two or more.

Examples of the alkaline-soluble resin include a methacrylic acid/benzylmethacrylate copolymer, a methacrylic acid/benzylmethacrylate/styrene copolymer, a methacrylic acid/benzylmethacrylate/2-hydroxyethylmethacrylate copolymer, a methacrylic acid/benzylmethacrylate/styrene/2-hydroxyethylmethacrylate copolymer, and the like, but are not limited thereto, and these may be used singularly or in combination of two or more.

The alkaline-soluble resin may have a weight average molecular weight ranging from about 3,000 to about 50,000, and in one embodiment, may have a weight average molecular weight ranging from about 5,000 to about 40,000. When the alkaline-soluble resin has a weight average molecular weight within the range, the close contacting property between the substrate is improved and excellent physical and chemical properties and adequate viscosity are obtained.

The alkaline-soluble resin included in the photosensitive resin composition is a main factor to affect the resolution of a pixel. As for the methacrylic acid/benzylmethacrylate copolymer, the resolution of a pixel is remarkably changed according to an acid value and a weight average molecular weight. When the methacrylic acid/benzyl methacrylate copolymer is included in a ratio of 25/75 (w/w), the acid value ranges from about 80 to about 120 mgKOH/g, and the weight average molecular weight ranges from about 20,000 to about 40,000, high resolution of a pixel may be obtained.

The alkaline-soluble resin may be included in an amount ranging from 0.5 to about wt % based on the total amount of the photosensitive resin composition, and in one embodiment, it may be included in an amount ranging from about 1 to about 20 wt % based on the total amount of the photosensitive resin composition. When the alkaline-soluble resin is included within the range, development properties are improved and surface flatness and smoothness are obtained due to improved cross-linking during fabrication of a color filter.

( [symbol] ) Photopolymerization Initiator

The photopolymerization initiator is a generally-used photopolymerization initiator for fabricating a photosensitive resin composition, and is selected from an acetophenone-based compound, a benzophenone-based compound, a thioxanthone-based compound, a benzoin-based compound, a triazine-based compound, an oxime-based compound, or a combination thereof.

The acetophenone-based compound may include 2,2′-diethoxyacetophenone, 2,2′-dibutoxyacetophenone, 2-hydroxy-2-methylpropinophenone, p-t-butyltrichloroacetophenone, p-t-butyldichloroacetophenone, 4-chloroacetophenone, 2,2′-dichloro-4-phenoxyacetophenone, 2-methyl-1-(4-(methylthio)phenyl)-2-morpholinopropan-1-one, 2-benzyl-2-dimethylamino-1-(4-morpholinophenyl)-butan-1-one, and the like.

The benzophenone-based compound may include benzophenone, benzoyl benzoic acid, benzoyl benzoic acid methyl, 4-phenyl benzophenone, hydroxy benzophenone, acrylated benzophenone, 4,4′-bis(dimethyl amino)benzophenone, 4,4′-bis(diethylamino)benzophenone, 4,4′-dimethylaminobenzophenone,4,4′-dichlorobenzophenone, 3,3′-dimethyl-2-methoxybenzophenone, and the like.

The thioxanthone-based compound may include thioxanthone, 2-methylthioxanthone, isopropyl thioxanthone, 2,4-diethyl thioxanthone, 2,4-diisopropyl thioxanthone, 2-chlorothioxanthone, and the like.

The benzoin-based compound may include benzoin, benzoin methyl ether, benzoin ethyl ether, benzoin isopropyl ether, benzoin isobutyl ether, benzyldimethylketal, and the like.

The triazine-based compound may include 2,4,6-trichloro-s-triazine, 2-phenyl 4,6-bis(trichloromethyl)-s-triazine, 2-(3′,4′-dimethoxystyryl)-4,6-bis(trichloromethyl)-s-triazine, 2(4′-methoxynaphthyl)-4,6-bis(trichloromethyl)-s-triazine, 2-(p-methoxyphenyl)-4,6-bis(trichloromethyl)-s-triazine, 2-(p-tolyl)-4,6-bis(trichloro methyl)-s-triazine, 2-biphenyl 4,6-bis(trichloro methyl)-s-triazine, bis(trichloromethyl)-6-styryl-s-triazine, 2-(naphtol-yl)-4,6-bis(trichloromethyl)-s-triazine, 2-(4-methoxynaphtol-yl)-4,6-bis(trichloromethyl)-s-triazine, 2-4-trichloromethyl(piperonyl)-6-triazine, 2-4-trichloromethyl(4′-methoxystyryl)-6-triazine, and the like.

The oxime-based compound may include 2-(o-benzoyloxime)-1-[4-(phenylthio)phenyl]-1,2-octanedione, 1-(o-acetyloxime)-1-[9-ethyl-6-(2-methylbenzoyl)-9H-carbazol-3-yl]ethaneone, and the like.

In addition to the photopolymerization initiator mentioned above, a carbazole-based compound, a diketone-based compound, a sulfonium borate-based compound, a diazo-based compound, and a non-imidazole-based compound may be used as a photopolymerization initiator.

The photopolymerization initiator may be included in an amount ranging from about 0.1 to about 10 wt % based on the total amount of the photosensitive resin composition, and in one embodiment, it may be included in an amount ranging from about 1 to about 5 wt % based on the total amount of the photosensitive resin composition.

When the photopolymerization initiator is included within the range, it may have enough photopolymerization during exposure through a pattern forming process for fabricating a color filter, and therefore excellent sensitivity and increased transmittance are acquired.

( [symbol] ) Photopolymerizable Monomer

The photopolymerizable monomer may include a multi-functional group monomer including at least 2 or more hydroxy groups. Examples of the photopolymerizable monomer include glycerolacrylate, dipentaerythritolhexaacrylate, ethyleneglycoldiacrylate, triethyleneglycoldiacrylate, 1,4-butanedioldiacrylate, 1,6-hexanedioldiacrylate, neopentylglycoldiacrylate, pentaerythritoldiacrylate, pentaerythritoltriacrylate, pentaerythritol diacrylate, dipentaerythritoltriacrylate, dipentaerythritolacrylate, pentaerythritolhexaacrylate, bisphenol A diacrylate, trimethylolpropanetriacrylate, novolac epoxy acrylate, ethyleneglycoldimethacrylate, diethyleneglycoldimethacrylate, triethyleneglycoldimethacrylate, propyleneglycoldimethacrylate, 1,4-butanedioldimethacrylate, 1,6-hexanedioldimethacrylate, and the like.

The photopolymerizable monomer may be included in an amount ranging from about 0.5 to about 30 wt % based on the total amount of photosensitive resin composition, and in one embodiment, it may be included in an amount ranging from about 1 to about 15 wt % based on the total amount of photosensitive resin composition. When the photopolymerizable monomer is included within the range, a pattern characteristic and development properties for fabricating a color filter are improved.

( [symbol] ) Solvent

As for the solvent, a solvent the same as the solvent ( [symbol] ) included in the pigment dispersion composition may be used.

The solvent may be included in a balance amount based on the total amount of the photosensitive resin composition, and in one embodiment, may be included in a range from about 20 to about 80 wt % based on the total amount of the photosensitive resin composition. When the solvent is included within the range, a viscosity of the photosensitive resin composition may be suitably maintained, and therefore physical and optical characteristics of articles may be improved when being applicable to the articles.

( [symbol] ) Other Additives

In order to prevent a stain or a spot, or a residue caused by underdevelopment during a coating process, and in order to have a leveling property, the photosensitive resin composition may include malonic acid, 3-amino-1,2-propanediol, a silane-based coupling agent including a vinyl group or a (meth)acryloxy group, and other additives such as a surfactant. The use amount of the additives may be controlled according to the physical properties.

In order to improve adherence and other characteristics, an epoxy compound may be further included in the photosensitive resin composition as needed.

The epoxy compound may be an epoxy novolac acryl carboxylate resin, an ortho cresol novolac epoxy resin, a phenol novolac epoxy resin, a tetramethyl biphenyl epoxy resin, a bisphenol A-type epoxy resin, an alicyclic epoxy resin, or a combination thereof.

The epoxy compound may be used in an amount ranging from 0.01 to 5 parts by weight based on 100 parts by weight of the photosensitive resin composition. When the epoxy compound is included within the range, it is possible to economically improve the storage property, the close contacting property, and other characteristics.

When more epoxy compound is included, a radical polymerization initiator such as a peroxide initiator or an azobis-based initiator may be further included.

There are no particular limits for a method of preparing the photosensitive resin composition, but in one embodiment, a photosensitive resin composition may be prepared by mixing a photopolymerizable monomer, a photopolymerization initiator, an alkaline-soluble resin, a solvent, and selectively an additive in the pigment dispersion composition.

According to another embodiment, a color filter fabricated by the photosensitive resin composition is provided.

The color filter may be fabricated by a general method. In one embodiment, the photosensitive resin composition is coated using a suitable method such as spin coating or slit coating on a glass with a thickness ranging from about 1.4 to about 3.3 μm. Ultraviolet (UV) rays are exposed on a coating layer to form a pattern for a color filter, which is then treated with an alkaline development solution on a coating layer and then a required pattern on an image color filter is formed after an unexposed part of the coating layer is dissolved. In order to obtain a color filter having a desired pattern, the process for forming a pattern is repeatedly performed according to the numbers of R, G, and B colors. Furthermore, the processes such as curing the obtained image pattern with heat or exposing actinic rays may be further performed in order to improve crack resistance and solvent resistance.

### MODE FOR THE INVENTION

Hereinafter, the present invention is illustrated in more detail with reference to examples. However, they are exemplary embodiments and are not limiting.

### Example 1-1

### Preparation of Pigment Dispersant

(1) Preparation of Pigment Affinity Moiety.

7.4 g (0.04 mol) of 2,4,6-trichlorotriazine and 11.9 g (0.08 mol) of 5-aminobenzoimidazolone were added to 240.0 g of 10 wt % of acetic acid dissolved in an aqueous solution in a reactor mounted with an agitator and reacted at a temperature of 10° C. for 1 hour, then, 32.0 g of a 10 wt % sodium hydroxide aqueous solution was added and agitated for 30 minutes, and filtered and washed to complete the first reaction ( [symbol] ). 16.36 g (0.04 mol) of the first reaction product obtained through the first reaction was added to the reactor with an agitator and agitated, and 9.0 g (0.06 mol) of p-aminoacetoanilide was added and reacted at a temperature of 80° C. for 2 hours, and then 60 g of a 37 wt % HCl aqueous solution was added and refluxed at a temperature of 90° C. for 4 hours. The resultant was cooled to 20° C., and a 10 wt % sodium hydroxide aqueous solution was slowly added until the pH was 9.5, and then filtered and dried using distilled water. Thereby, a reddish brown powder was obtained and the second reaction ( [symbol] ) was completed. 24.1 g (0.05 mol) of the second reaction product obtained through the second reaction and 10.9 g (0.05 mol) of pyromellitic dianhydride were added to 150 g of ethylether and reacted at room temperature for 5 hours, and then filtered and dried to complete the third reaction ( [symbol] ). Thereby, a pigment affinity moiety was prepared by obtaining 25.5 g of the third reaction product obtained through the third reaction.

(2) Preparation of Solvent Affinity Moiety

10.0 g of 1-dodecanol, 98.0 g of ε-caprolactone, and 0.03 g of dibutyltin (IV) oxide as a catalyst were added to another reactor with an agitator and heated to 140° C. and then reacted for 5 hours. Thereby, a wax-type material was obtained. A solvent affinity moiety was prepared by finishing the reaction after confirming 97.3 wt % of the reactants was reacted using a solid measurement.

(3) Preparation of Pigment Dispersant

108 g (0.054 mol) of a caprolactone resin of the solvent affinity moiety and 19.4 g (0.027 mol) of the third reaction product of the pigment affinity moiety were added to 0.05 g of dibutyltin (IV) oxide dissolved in 300 g of cyclohexanone and agitated. Then, the resultant was reacted at a temperature of 140° C. for 7 hours to prepare a pigment dispersant having a weight average molecular weight of 4850 and a number average molecular weight of 3345, measured by GPC.

### Examples 1-2 TO 1-14

### Preparation of Pigment Dispersant

Each of the pigment dispersants according to Examples 1-2 to 1-14 was prepared according to the compound and the amount of the following Table 1, using the same reactor, catalyst, and reaction operation as Example 1-1.

TABLE 1

| | | | Examples | | | | | | | |
|---|---|---|---|---|---|---|---|---|---|---|
| | | | 1-1 | 1-2 | 1-3 | 1-4 | 1-5 | 1-6 | 1-7 | 1-8 |
| Pigment | First | 2,4,6- | 7.4 | 7.4 | 7.4 | 7.4 | — | 7.4 | 7.4 | 7.4 |
| affinity | reaction | trichlorotriazine (g) | | | | | | | | |
| moiety | | 5-aminobenzo- | 11.9 | — | — | — | — | 11.9 | 11.9 | 11.9 |
| | | imidazolone (g) | | | | | | | | |
| | | 3-aminopropanol (g) | — | 6 | — | — | — | — | — | — |
| | | Aniline (g) | — | — | 7.4 | — | — | — | — | — |
| | | Diethylamino- | — | — | — | 10.4 | — | — | — | — |
| | | propylamine (g) | | | | | | | | |
| | | Diethylamine (g) | — | — | — | — | 5.84 | — | — | — |
| | | First reaction | 16.36 | 10.4 | 11.9 | 14.8 | 11.4 | 12.8 | 12.8 | 12.8 |
| | | product (g) | | | | | | | | |
| | Second | ρ-aminoaceto- | 9 | 9 | 9 | 9 | 9 | 9 | 9 | 9 |
| | reaction | anilide (g) | | | | | | | | |
| | | Second reaction | 24.1 | 16.7 | 18.5 | 22.1 | 17.9 | 24.1 | 24.1 | 24.1 |
| | | product (g) | | | | | | | | |
| | Third | Pymellitic | 10.9 | 10.9 | 10.9 | 10.9 | 10.9 | — | — | — |
| | reaction | dianhydride (g) | | | | | | | | |
| | | Biphenyltetra- | — | — | — | — | — | 14.7 | — | — |
| | | carboxylic acid | | | | | | | | |
| | | dianhydride (g) | | | | | | | | |
| | | Cyclobutane- | — | — | — | — | — | — | 9.8 | — |
| | | tetracarboxylic | | | | | | | | |
| | | dianhydride (g) | | | | | | | | |
| | | Naphthalene- | — | — | — | — | — | — | — | 13.4 |
| | | tetracarboxylic | | | | | | | | |
| | | acid dianhydride (g) | | | | | | | | |
| | | Benzophenone | — | — | — | — | — | — | — | — |
| | | tetracarboxylic | | | | | | | | |
| | | dianhydride (g) | | | | | | | | |
| | | Third reaction | 19.4 | 15.4 | 16.4 | 18.3 | 16 | 21.4 | 18.8 | 20.7 |
| | | product (g) | | | | | | | | |

| | | | | | | | | | |
|---|---|---|---|---|---|---|---|---|---|
| Solvent | 1-dodecanol (g) | 10 | 10 | 10 | 10 | 10 | 10 | 10 | 10 |
| affinity | Cetanol (g) | — | — | — | — | — | — | — | — |
| moiety | Octadecyl | — | — | — | — | — | — | — | — |
| | alcohol (g) | | | | | | | | |
| | Caprolactone | 98.5 | 98.5 | 98.5 | 98.5 | 98.5 | 98.5 | 98.5 | 98.5 |
| | resin (g) | | | | | | | | |
| | Dibutyltin (IV) | 0.09 | 0.09 | 0.09 | 0.09 | 0.09 | 0.09 | 0.09 | 0.09 |
| | oxide (g) | | | | | | | | |

| | | | | | | | | |
|---|---|---|---|---|---|---|---|---|
| Weight average | 4850 | 3894 | 4265 | 4562 | 4321 | 4921 | 4423 | 5062 |
| molecular weight | | | | | | | | |
| Number average | 3345 | 2863 | 2682 | 2765 | 3043 | 2704 | 3655 | 3330 |
| molecular weight | | | | | | | | |

| | | | Examples | | | | | |
|---|---|---|---|---|---|---|---|---|
| | | | 1-9 | 1-10 | 1-11 | 1-12 | 1-13 | 1-14 |
| Pigment | First | 2,4,6- | 7.4 | 7.4 | 7.4 | 7.4 | 7.4 | 7.4 |
| affinity | reaction | trichlorotriazine (g) | | | | | | |
| moiety | | 5-aminobenzo- | 11.9 | 11.9 | 11.9 | 11.9 | 11.9 | 11.9 |
| | | imidazolone (g) | | | | | | |
| | | 3-aminopropanol (g) | — | — | — | — | — | — |
| | | Aniline (g) | — | — | — | — | — | — |
| | | Diethylamino- | — | — | — | — | — | — |
| | | propylamine (g) | | | | | | |
| | | Diethylamine (g) | — | — | — | — | — | — |
| | | First reaction | 12.8 | 12.8 | 12.8 | 16.36 | 16.36 | 16.36 |
| | | product (g) | | | | | | |
| | Second | ρ-aminoaceto- | 9 | 9 | 9 | 9 | 9 | 9 |
| | reaction | anilide (g) | | | | | | |
| | | Second reaction | 24.1 | — | — | 24.1 | 24.1 | 24.1 |
| | | product (g) | | | | | | |
| | Third | Pymellitic | — | — | — | 10.9 | 10.9 | 10.9 |
| | reaction | dianhydride (g) | | | | | | |
| | | Biphenyltetra- | — | — | — | — | — | — |
| | | carboxylic acid | | | | | | |
| | | dianhydride (g) | | | | | | |
| | | Cyclobutane- | — | — | — | — | — | — |
| | | tetracarboxylic | | | | | | |
| | | dianhydride (g) | | | | | | |
| | | Naphthalene- | — | — | — | — | — | — |
| | | tetracarboxylic | | | | | | |
| | | acid dianhydride (g) | | | | | | |
| | | Benzophenone | 16.1 | — | — | — | — | — |
| | | tetracarboxylic | | | | | | |
| | | dianhydride (g) | | | | | | |
| | | Third reaction | 22.2 | — | — | 19.4 | 19.4 | 19.4 |
| | | product (g) | | | | | | |

| | | | | | | | |
|---|---|---|---|---|---|---|---|
| Solvent | 1-dodecanol (g) | 10 | — | — | — | 10 | 10 |
| affinity | Cetanol (g) | — | 13.1 | — | — | — | — |
| moiety | Octadecyl | — | — | 14.6 | — | — | — |
| | alcohol (g) | | | | | | |
| | Caprolactone | 98.5 | 73.9 | 73.9 | 73.9 | 49.3 | 24.6 |
| | resin (g) | | | | | | |
| | Dibutyltin (IV) | 0.09 | 0.09 | 0.09 | 0.09 | 0.09 | 0.09 |
| | oxide (g) | | | | | | |

| | | | | | | |
|---|---|---|---|---|---|---|
| Weight average | 5166 | 3521 | 3654 | 3345 | 2364 | 1423 |
| molecular weight | | | | | | |
| Number average | 3690 | 2910 | 2487 | 2451 | 1630 | 1003 |
| molecular weight | | | | | | |

### Example 2

### Preparation of Pigment Dispersant

(1) Preparation of Pigment Affinity Moiety

14.6 g (0.05 mol) of a pigment red 255 (Scarlet EK) and 150 g of sulfuric chloride were agitated at a temperature of 55° C. for 2 hours in a reactor with a agitator and completely dissolved. The resultants were cooled to room temperature and 17.9 g of thionyl chloride was added, then heated again to 55° C. and agitated for 1 hour, dipped in an ice-supported aqueous solution, and then filtered to complete the first reaction ( [symbol] ). 19.3 g of the first reaction product obtained through was added to 200 g of an aqueous solution including 9.0 g (0.06 mol) of p-aminoacetoanilide, and agitated at a temperature of 10° C. for 2 hours, reacted at a temperature of 80° C. for 2 hours, and then 60 g of a 37 wt % HCl aqueous solution was added and refluxed at a temperature of 90° C. for 4 hours. The resultant was cooled to 20° C., and a 10 wt % sodium hydroxide aqueous solution was slowly added until the pH was 9.5, and then filtered and dried using distilled water, and thereby a red powder was obtained and the second reaction ( [symbol] ) was completed. 22.9 g (0.05 mol) of the second reaction product obtained through the second reaction and 10.9 g (0.05 mol) of pyromellitic dianhydride were added to 150 g of ethylether and reacted at room temperature for 5 hours, and then filtered and dried complete the third reaction ( [symbol] ). Thereby, a pigment affinity moiety was prepared by obtaining the third reaction product obtained through the third reaction.

(2) Preparation of Solvent Affinity Moiety

10.0 g of 1-dodecanol, 98.0 g of ε-caprolactone, and 0.03 g of dibutyltin (IV) oxide as a catalyst were added to another reactor with an agitator, and heated to 140° C. then reacted for 5 hours, and a wax-type material was obtained. A solvent affinity moiety was prepared by finishing the reaction above after confirming 97.3 wt % of the reactants above were reacted using a solid measurement.

(3) Preparation of Pigment Dispersant

108 g (0.054 mol) of a caprolactone resin of the solvent affinity moiety and 19.4 g (0.027 mol) of the third reaction product of the pigment affinity moiety were added to 300 g of cyclohexanone with 0.05 g of dibutyltin (IV) oxide dissolved therein, and agitated. Then, the resultant was reacted at a temperature of 140° C. for 7 hours to prepare a light red pigment dispersant having a weight average molecular weight of 5262, measured by GPC.

### Example 3

### Preparation of Pigment Dispersant

A pigment dispersant was prepared according to the same method as in Example 2, except that pigment yellow 138 was used instead of pigment red 255 for preparing a pigment affinity moiety of Example 2. A yellow pigment dispersant having a molecular weight of 5740 measured by GPC was prepared.

### Example 4

### Preparation of Pigment Dispersant

A pigment dispersant was prepared according to the same method as Example 2, except that pigment blue 15-6 was used instead of pigment red 255 for preparing a pigment affinity moiety of Example 2. A purple pigment dispersant having a molecular weight of 4240 measured by GPC was prepared.

### Example 5

### Preparation of Pigment Dispersion Composition

10.0 g of the pigment dispersant prepared from Example 1-1 and 9.0 g of a binder resin obtained by copolymerization of benzylmethacrylate and methacrylic acid with a 8:2 ratio and having a weight average molecular weight of 20,000 were dissolved in 151.0 g of propyleneglycolmethylether as a solvent and agitated, then 16.0 g of a pigment green 58 (manufactured by DIC Co., KE005) and 14.0 g of a pigment yellow 150 (manufactured by DNS Co., TDY5) were added as a pigment and agitated and dispersed for 5 hours using a pico dispenser including a dispersion chamber of 50 mL and 75 volume % charged with 0.1 mm zirconia beads, and thereby a green pigment dispersion composition having a viscosity of 7.51 cP was prepared.

### Examples 6 TO 9 and Comparative Example 1

### Preparation of Pigment Dispersion Composition

Each pigment dispersion composition of Examples 6 to 9 and Comparative Example 1 was prepared by the reaction using the compound and amount represented by the following Table 2 and using the same disperser, the same process for reaction, and the same reaction time as in Example 5.

In Example 9, pigment red 177(DNS Co., TDR2) and pigment red 254 (DNS Co., TDR4) were used as a pigment.

In Comparative Example 1, BYK2001 manufactured by BYK Corp. was used as a pigment dispersant.

### Experimental Example 1

### Stability Evaluation of Pigment Dispersion Composition

Stability of the pigment dispersion compositions prepared according to Examples 5 to 9 and Comparative Example 1 was evaluated by measuring a viscosity, and the results are shown in the following Table 2.

The viscosity was measured with a DVII Ultra (manufactured by Brookfield Co.) under a 45 to 50% torque variation ratio (%) at 25° C., and the initial viscosity and the viscosity measured after 7 days from obtaining of pigment dispersion compositions are shown in Table 2.

TABLE 2

| | | Ex. | | | | | Comp. |
|---|---|---|---|---|---|---|---|
| | | 5 | 6 | 7 | 8 | 9 | Ex. 1 |
| Pigment | Pigment green 58 (g) | 16.0 | 16.0 | 16.0 | 16.0 | — | 16.0 |
| | Pigment red 177 (g) | — | — | — | — | 4.0 | — |
| | Pigment red 254 (g) | — | — | — | — | 4.0 | — |
| | Pigment yellow 150 (g) | 14.0 | 14.0 | 14.0 | 14.0 | 2.0 | 14.0 |
| Pigment | Dispersing agent (g) prepared by | 10.0 | — | 6.0 | 5.0 | 6.0 | |
| dispersant | Example 1-1 | | | | | | |
| | Dispersing agent (g) prepared by | — | 10.0 | — | — | — | — |
| | Example 1-2 | | | | | | |
| | Dispersing agent (g) prepared by | — | — | — | — | 4.0 | — |
| | Example 2 | | | | | | |
| | Dispersing agent (g) prepared by | — | — | 4.0 | 2.0 | — | — |
| | Example 3 | | | | | | |
| | Dispersing agent (g) prepared by | — | — | — | 3.0 | — | — |
| | Example 4 | | | | | | |
| | BYK2001 (g) | — | — | — | — | — | 10.0 |

| | | | | | | |
|---|---|---|---|---|---|---|
| Binder resin (g) | 9.0 | 9.0 | 9.0 | 9.0 | 9.0 | 9.0 |
| Solvent (g) | 151.0 | 151.0 | 151.0 | 151.0 | 151.0 | 151.0 |
| Initial viscosity (cP) | 7.51 | 9.54 | 6.51 | 6.24 | 15.2 | 23.4 |
| Viscosity (cP) after 7 days | 7.68 | 9.85 | 6.85 | 6.39 | 16.5 | 52.3 |
| Variation ratio (%) | 2.26 | 3.24 | 5.22 | 2.40 | 8.55 | 123.5 |

As shown in Table 2, the pigment dispersion compositions of Examples 5 to 9 prepared using the compound according to one embodiment as a pigment dispersant have viscosity variation ratios within 10% after 7 days, indicating that they have excellent stability compared with Comparative Example 1.

### Examples 10 TO 12 AND COMPARATIVE EXAMPLE 2

### Preparation of Photosensitive Resin Composition

1-piperonyl-4,6-bis(trichloromethyl)-1,3,5-triazine was dissolved in a solvent as a photopolymerization initiator and agitated for 2 hours, then a methacrylic acid/benzylmethacrylate copolymer (Mw=20,000) as a alkaline-soluble resin and dipentaerythritol hexaacrylate as a photopolymerizable monomer were added and agitated at room temperature for 2 hours. The pigment dispersion compositions according to Table 3 were respectively added and agitated at room temperature for 1 hour, and then F475 (manufactured by DIC Co.) was added as a surfactant and agitated at room temperature for 1 hour. Each of the resultants was filtered 3 times to remove impurities, and thereby photosensitive resin compositions according to Examples 10 to 12 and Comparative Example 2 were prepared according to Table 3.

### Experimental Example 2

### Color Characteristics and Developing Time of a Photosensitive Resin Composition

The color characteristics (luminance and contrast ratio) and the development time of the photosensitive resin compositions prepared according to Examples 10 to 12 and Comparative Example 2 were evaluated, and the results are shown in Table 3.

The photosensitive resin compositions of Examples 10 to 12 and Comparative Example 2 were respectively coated with a thickness of 2.0 to 2.5 μm on bare glass with a spin coater. The resultant was soft-baked using a hot plate at 80° C. for 150 seconds. Then, it was exposed to 60 mJ power using an exposer, developed for 60 seconds and washed for 60 seconds using a developer, and spin-dried for 25 seconds. Herein, luminance was measured using a spectrophotometer (manufactured by Otsuka Co., Ltd., MCPD-3000). The contrast ratio is obtained by mounting a glass substrate between two polarizers and then measuring a ratio between parallel and vertical polarizers using a contrast tester (manufactured by Tsubosaka electronic Co., Ltd, CT-1).

The development time was measured by developing using a 1% potassium hydroxide-based development solution at 25° C. Next, hard-baking was performed in an oven at 230° C. for 30 minutes. The hard baked specimen was measured using a color meter.

TABLE 3

| | | | | | Comp. |
|---|---|---|---|---|---|
| | | Ex. 10 | Ex. 11 | Ex. 12 | Ex 2 |
| Alkaline-soluble resin (g) | | 9.9 | 9.9 | 9.9 | 9.9 |
| Photopolymerizable | | 4.1 | 4.1 | 4.1 | 4.1 |
| monomer (g) | | | | | |
| Photopolymerization | | 0.2 | 0.2 | 0.2 | 0.2 |
| initiator (g) | | | | | |
| Pigment | Example 5 | 37.9 | — | — | — |
| dispersion | Example 6 | — | 37.9 | — | — |
| com- | Example 7 | — | — | 37.9 | — |
| position | Comparative | — | — | — | 37.9 |
| | Example 1 | | | | |
| Solvent | Propylene | 35 | 35 | 35 | 35 |
| | glycol | | | | |
| | methylether | | | | |
| | (g) | | | | |
| | 2-ethoxy | 12.8 | 12.8 | 12.8 | 12.8 |
| | ethyl | | | | |
| | propionate | | | | |
| | (g) | | | | |

| | | | | |
|---|---|---|---|---|
| Surfactant (g) | 0.1 | 0.1 | 0.1 | 0.1 |
| Gx | 0.301 | 0.303 | 0.299 | 0.304 |
| Gy | 0.595 | 0.595 | 0.595 | 0.595 |
| Luminance (Y) | 59.5 | 59.4 | 58.7 | 58.2 |
| Contrast ratio (C/R) | 12500 | 11400 | 10900 | 7500 |
| Development time (BP) | 31 | 34 | 41 | 50 |
| (s) | | | | |

As shown in Table 3, the photosensitive resin compositions of Examples 5 to 9 prepared using the compounds prepared according to one embodiment as a pigment dispersant have increased luminance and contrast ratio, and a decreased development time compared with Comparative Example 2.

While this disclosure has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be exemplary but not limiting the present invention in any way.

### INDUSTRIAL APPLICABILITY

The novel compound can be used in a pigment dispersion composition having high dispersibility and storage stability in a small amount.

1. A compound represented by the following Chemical Formula 1: 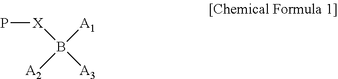
wherein, in Chemical Formula 1,
B is a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, or a substituted or unsubstituted C6 to C30 arylene group,
A₁ to A₃ are independently a substituent represented by the following Chemical Formula 3, CH₂COOH, COOH, or SO₃H,
X is CONH, and
P is one of substituents represented by the following Chemical Formulae 4-1 to 4-4, 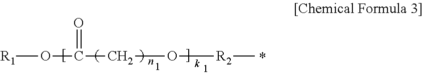
wherein, in Chemical Formula 3,
R₁ is a substituent derived from a compound including a hydroxy group,
R₂ is CO or SO₂,
n₁ is an integer ranging from 1 to 6, and
k₁ is an integer ranging from 1 to 20, 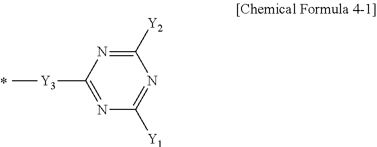
wherein, in Chemical Formula 4-1,
Y₁ and Y₂ are independently hydrogen, a halogen, a hydroxy group, a substituted or unsubstituted C1 to C20 alkoxy group, a substituted or unsubstituted C1 to C20 alkyl group, a substituted or unsubstituted C2 to C20 alkenyl group, a substituted or unsubstituted C2 to C20 alkynyl group, a substituted or unsubstituted C3 to C20 cycloalkyl group, a substituted or unsubstituted C3 to C20 cycloalkenyl group, a substituted or unsubstituted C3 to C20 cycloalkynyl group, a substituted or unsubstituted C2 to C20 heterocycloalkyl group, a substituted or unsubstituted C2 to C20 heterocycloalkenyl group, a substituted or unsubstituted C2 to C20 heterocycloalkynyl group, a substituted or unsubstituted C6 to C30 aryl group, a substituted or unsubstituted C6 to C30 aryloxy group, an amine group including a substituted or unsubstituted C1 to C20 alkyl group or a C6 to C30 aryl group, or an amine group including a substituted or unsubstituted C1 to C20 alkyl group or a C6 to C30 aryl group, and
Y₃ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group, 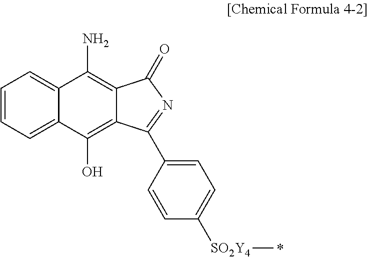
wherein, in Chemical Formula 4-2,
Y₄ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group, 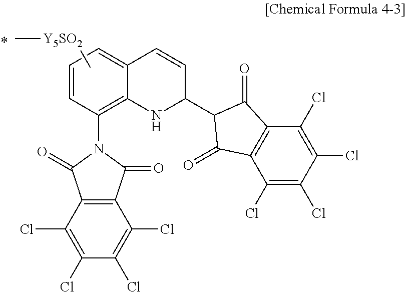
wherein, in Chemical Formula 4-3,
Y₅ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group, and 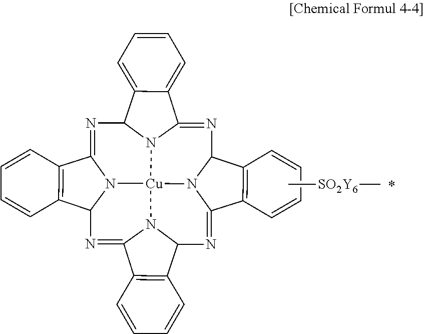
wherein, in Chemical Formula 4-4,
Y₆ is hydrogen, a halogen, a hydroxy group, a substituted or unsubstituted C1 to C20 oxyalkylene group, a substituted or unsubstituted C1 to C20 alkylene group, a substituted or unsubstituted C2 to C20 alkenylene group, a substituted or unsubstituted C2 to C20 alkynylene group, a substituted or unsubstituted C3 to C20 cycloalkylene group, a substituted or unsubstituted C3 to C20 cycloalkenylene group, a substituted or unsubstituted C3 to C20 cycloalkynylene group, a substituted or unsubstituted C2 to C20 heterocycloalkylene group, a substituted or unsubstituted C2 to C20 heterocycloalkenylene group, a substituted or unsubstituted C2 to C20 heterocycloalkynylene group, a substituted or unsubstituted C6 to C30 arylene group, a substituted or unsubstituted C6 to C30 oxyarylene group, an amine group including a substituted or unsubstituted C1 to C20 alkyl group or a C6 to C30 aryl group, or an amine group including a substituted or unsubstituted C1 to C20 alkyl group or a C6 to C30 aryl group.

2. The compound of claim 1, wherein the compound of Chemical Formula 1 has a weight average molecular weight ranging from 3,000 to 15,000.

3. A pigment dispersion composition, comprising (A) a pigment, (B) a pigment dispersant including the compound of claim 1, (C) a binder resin, and (D) a solvent.

4. A pigment dispersion composition of claim 3, wherein the pigment dispersion composition comprises 5 to 40 wt % of the pigment (A), 1 to 20 wt % of the pigment dispersant (B), 1 to 20 wt % of the binder resin (C), and a balance amount of the solvent (D).

5. A pigment dispersion composition of claim 3, wherein the pigment dispersion composition has a viscosity ranging from 1 to 50 cps.

6. A photosensitive resin composition comprising the pigment dispersion composition of any one of claims 3 to 5.

7. A color filter fabricated using the photosensitive resin composition of claim 6.
